SEQUENCE LISTING (74 sequences)

SEQ ID NO: 1 - protein, 123 aa, synthetic construct
  REGION 1..123: note = AR8804
AVQLVDSGGGLVQAGGSLRLSCAASGSSVSRYAMAWYRQAPGKERELVAVVRITGIPIYADFVKGRFTISRDNAKNTVLLQMNSLEPEDTAIYYCNTGNFNTYTRRYSPSETWGQGTQVTVSS

SEQ ID NO: 2 - protein, 5 aa, synthetic construct
  REGION 1..5: note = CDR1 of AR8804
RYAMA

SEQ ID NO: 3 - protein, 16 aa, synthetic construct
  REGION 1..16: note = CDR2 of AR8804
VVRITGIPIYADFVKG

SEQ ID NO: 4 - protein, 15 aa, synthetic construct
  REGION 1..15: note = CDR3 of AR8804
GNFNTYTRRYSPSET

SEQ ID NO: 5 - protein, 123 aa, synthetic construct
  REGION 1..123: note = AR8820
QVKLEESGGGLVQAGGSLRLSCAASGSSISRYAMAWYRQGPGKERELVAVVRITGSPIYADFVGGRFTISRDNAKSTVLLQMNSLKPEDTAIYYCNTGNFNTYTRRYSPSETWGQGTQVTVSS

SEQ ID NO: 6 - protein, 5 aa, synthetic construct
  REGION 1..5: note = CDR1 of AR8820
RYAMA

SEQ ID NO: 7 - protein, 16 aa, synthetic construct
  REGION 1..16: note = CDR2 of AR8820
VVRITGSPIYADFVGG

SEQ ID NO: 8 - protein, 15 aa, synthetic construct
  REGION 1..15: note = CDR3 of AR8820
GNFNTYTRRYSPSET

SEQ ID NO: 9 - protein, 121 aa, synthetic construct
  REGION 1..121: note = AR8776
AVQLVESGGGLVQPGGSLRLSCAASGFTFSNYAMSWVRQAPGKGLEWVSSINRGGSITNYADSVKGRFAISRDNAKNTIYLQMNNLRPEDTAQYSCARGDSTYSRLSAAYRGQGTQVTVSS

SEQ ID NO: 10 - protein, 5 aa, synthetic construct
  REGION 1..5: note = CDR1 of AR8776
NYAMS

SEQ ID NO: 11 - protein, 17 aa, synthetic construct
  REGION 1..17: note = CDR2 of AR8776
SINRGGSITNYADSVKG

SEQ ID NO: 12 - protein, 12 aa, synthetic construct
  REGION 1..12: note = CDR3 of AR8776
GDSTYSRLSAAY

SEQ ID NO: 13 - protein, 121 aa, synthetic construct
  REGION 1..121: note = AR8875
QVQLVESGGGLVQPGGSLRLSCAASGFTFSNYAMTWVRQAPGKGLEWVSSIDRGGSITSYADSVKGRFAISRDNAKNTMYLQMNNLRPEDTARYVCARGDSTYSRLTAAYRGQGTQVTVSS

SEQ ID NO: 14 - protein, 5 aa, synthetic construct
  REGION 1..5: note = CDR1 of AR8775
NYAMT

SEQ ID NO: 15 - protein, 17 aa, synthetic construct
  REGION 1..17: note = CDR2 of AR8775
SIDRGGSITSYADSVKG

SEQ ID NO: 16 - protein, 12 aa, synthetic construct
  REGION 1..12: note = CDR3 of AR8775
GDSTYSRLTAAY

SEQ ID NO: 17 - protein, 121 aa, synthetic construct
  REGION 1..121: note = AR8866
QVKLEESGGGLVQPGGSLRLSCVASGFTFSVWTMSWVRQAPGKGLEWVSDISHSGIYTTYADSVKGRFTISRDNAKNTLWLQLNALKPEDTAVYYCAQGNRRNSMLSGTLRGQGTQVTVSS

SEQ ID NO: 18 - protein, 5 aa, synthetic construct
  REGION 1..5: note = CDR1 of AR8866
VWTMS

SEQ ID NO: 19 - protein, 17 aa, synthetic construct
  REGION 1..17: note = CDR2 of AR8866
DISHSGIYTTYADSVKG

SEQ ID NO: 20 - protein, 12 aa, synthetic construct
  REGION 1..12: note = CDR3 of AR8866
GNRRNSMLSGTL

SEQ ID NO: 21 - protein, 124 aa, synthetic construct
  REGION 1..124: note = AR8887
QVQLVESGGGLVQAGGSLRLSCAVSGFLYYSIGWFRQAPGKEREWVSCISRSGAGTSYANSVKGRSTISRDNDKNTVYLQMNSLKSEDTAVYYCARRATSSCTATMTRGQYDYWGQGTQVTVSS

SEQ ID NO: 22 - protein, 4 aa, synthetic construct
  REGION 1..4: note = CDR1 of AR8887
YSIG

SEQ ID NO: 23 - protein, 17 aa, synthetic construct
  REGION 1..17: note = CDR2 of AR8887
CISRSGAGTSYANSVKG

SEQ ID NO: 24 - protein, 17 aa, synthetic construct
  REGION 1..17: note = CDR3 of AR8887
RATSSCTATMTRGQYDY

SEQ ID NO: 25 - protein, 121 aa, synthetic construct
  REGION 1..121: note = AR8861
AVQLVDSGGGLVQPGGSLRLSCVASGFTFSIWTMSWVRQAPGKGLEWVSDISHSGLYTTYADSVKGRFTISRDNAKNTLWLQLNALKPEDTAVYYCAQGNRRTSMLSGTLRGQGTQVTVSS

SEQ ID NO: 26 - protein, 5 aa, synthetic construct
  REGION 1..5: note = CDR1 of AR8861
IWTMS

SEQ ID NO: 27 - protein, 17 aa, synthetic construct
  REGION 1..17: note = CDR2 of AR8861
DISHSGLYTTYADSVKG

SEQ ID NO: 28 - protein, 12 aa, synthetic construct
  REGION 1..12: note = CDR3 of AR8861
GNRRTSMLSGTL

SEQ ID NO: 29 - protein, 121 aa, synthetic construct
  REGION 1..121: note = AR8861-1.1
EVQLVESGGGLVQPGGSLRLSCAASGFTFSIWTMSWVRQAPGKGLEWVSDISHSGLYTTYADSVKGRFTISRDNSKNTLYLQMNSLRAEDTAVYYCAKGNRRTSMLSGTLWGQGTLVTVSS

SEQ ID NO: 30 - protein, 121 aa, synthetic construct
  REGION 1..121: note = AR8861-1.2
EVQLVESGGGLVQPGGSLRLSCAASGFTFSIWTMSWVRQAPGKGLEWVSDISHSGLYTTYADSVKGRFTISRDNSKNTLYLQMNSLRAEDTAVYYCAQGNRRTSMLSGTLWGQGTLVTVSS

SEQ ID NO: 31 - protein, 121 aa, synthetic construct
  REGION 1..121: note = AR8861-2.1
EVQLVESGGGLVKPGGSLRLSCAASGFTFSIWTMSWVRQAPGKGLEWVSDISHSGLYTTYADSVKGRFTISRDNAKNSLYLQMNSLRAEDTAVYYCARGNRRTSMLSGTLWGQGTLVTVSS

SEQ ID NO: 32 - protein, 121 aa, synthetic construct
  REGION 1..121: note = AR8861-2.2
EVQLVESGGGLVKPGGSLRLSCAASGFTFSIWTMSWVRQAPGKGLEWVSDISHSGLYTTYADSVKGRFTISRDNAKNSLYLQMNSLRAEDTAVYYCAQGNRRTSMLSGTLWGQGTLVTVSS

SEQ ID NO: 33 - protein, 121 aa, synthetic construct
  REGION 1..121: note = AR8861-3
EVQLVESGGGLVQPGGSLRLSCAASGFTFSIWTMSWVRQAPGKGLVWVSDISHSGLYTTYADSVKGRFTISRDNAKNTLYLQMNSLRAEDTAVYYCAQGNRRTSMLSGTLWGQGTLVTVSS

SEQ ID NO: 34 - protein, 121 aa, synthetic construct
  REGION 1..121: note = AR8861-4.1
EVQLVESGGGLVQPGGSLRLSCAASGFTFSIWTMSWVRQAPGKGLEWVSDISHSGLYTTYADSVKGRFTISRDNAKNSLYLQMNSLRAEDTAVYYCARGNRRTSMLSGTLWGQGTLVTVSS

SEQ ID NO: 35 - protein, 121 aa, synthetic construct
  REGION 1..121: note = AR8861-4.2
EVQLVESGGGLVQPGGSLRLSCAASGFTFSIWTMSWVRQAPGKGLEWVSDISHSGLYTTYADSVKGRFTISRDNAKNSLYLQMNSLRAEDTAVYYCAQGNRRTSMLSGTLWGQGTLVTVSS

SEQ ID NO: 36 - protein, 121 aa, synthetic construct
  REGION 1..121: note = AR8861-5.1
EVQLVESGGGLVQPGGSLRLSCAASGFTFSIWTMSWVRQAPGKGLEWVSDISHSGLYTTYADSVKGRFTISRDNSKNTLYLQMNSLRAEDTAVYYCARGNRRTSMLSGTLWGQGTLVTVSS

SEQ ID NO: 37 - protein, 121 aa, synthetic construct; identical to SEQ ID NO: 30
  REGION 1..121: note = AR8861-5.2

SEQ ID NO: 38 - protein, 121 aa, synthetic construct
  REGION 1..121: note = AR8861-6
QVQLVESGGGVVQPGRSLRLSCAASGFTFSIWTMSWVRQAPGKGLEWVADISHSGLYTTYADSVKGRFTISRDNSKNTLYLQMNSLRAEDTAVYYCAQGNRRTSMLSGTLWGQGTLVTVSS

SEQ ID NO: 39 - protein, 123 aa, synthetic construct
  REGION 1..123: note = AR8804-1.1
EVQLVESGGGLIQPGGSLRLSCAASGSSVSRYAMAWYRQAPGKGRELVSVVRITGIPIYADFVKGRFTISRDNSKNTLYLQMNSLRAEDTAVYYCATGNFNTYTRRYSPSETWGQGTTVTVSS

SEQ ID NO: 40 - protein, 123 aa, synthetic construct
  REGION 1..123: note = AR8804-1.3
EVQLVESGGGLIQPGGSLRLSCAASGSSVSRYAMAWYRQAPGKGRELVSVVRITGIPIYADFVKGRFTISRDNSKNTLYLQMNSLRAEDTAVYYCNTGNFNTYTRRYSPSETWGQGTTVTVSS

SEQ ID NO: 41 - protein, 123 aa, synthetic construct
  REGION 1..123: note = AR8804-2.3
EVQLVESGGGLVQPGGSLRLSCAASGSSVSRYAMAWVRQAPGKGRELVAVVRITGIPIYADFVKGRFTISRDNAKNSLYLQMNSLRAEDTAVYYCNTGNFNTYTRRYSPSETWGQGTLVTVSS

SEQ ID NO: 42 - protein, 123 aa, synthetic construct
  REGION 1..123: note = AR8804-3.1
QVQLVESGGGVVQPGGSLRLSCAASGSSVSRYAMAWYRQAPGKGRELVAVVRITGIPIYADFVKGRFTISRDNSKNTLYLQMNSLRAEDTAVYYCATGNFNTYTRRYSPSETWGKGTTVTVSS

SEQ ID NO: 43 - protein, 123 aa, synthetic construct
  REGION 1..123: note = AR8804-3.2
QVQLVESGGGVVQPGGSLRLSCAASGSSVSRYAMAWYRQAPGKGREWVAVVRITGIPIYADFVKGRFTISRDNSKNTLYLQMNSLRAEDTAVYYCATGNFNTYTRRYSPSETWGKGTTVTVSS

SEQ ID NO: 44 - protein, 123 aa, synthetic construct
  REGION 1..123: note = AR8804-3.3
QVQLVESGGGVVQPGGSLRLSCAASGSSVSRYAMAWYRQAPGKGRELVAVVRITGIPIYADFVKGRFTISRDNSKNTLYLQMNSLRAEDTAVYYCNTGNFNTYTRRYSPSETWGKGTTVTVSS

SEQ ID NO: 45 - protein, 123 aa, synthetic construct
  REGION 1..123: note = AR8820-1.1
QVQLVESGGGVVQPGGSLRLSCAASGSSISRYAMAWYRQAPGKGRELVAVVRITGSPIYADFVGGRFTISRDNSKNTLYLQMNSLRAEDTAVYYCATGNFNTYTRRYSPSETWGKGTTVTVSS

SEQ ID NO: 46 - protein, 123 aa, synthetic construct
  REGION 1..123: note = AR8820-1.2
QVQLVESGGGVVQPGGSLRLSCAASGSSISRYAMAWYRQAPGKGREWVAVVRITGSPIYADFVGGRFTISRDNSKNTLYLQMNSLRAEDTAVYYCATGNFNTYTRRYSPSETWGKGTTVTVSS

SEQ ID NO: 47 - protein, 123 aa, synthetic construct
  REGION 1..123: note = AR8820-1.3
QVQLVESGGGVVQPGGSLRLSCAASGSSISRYAMAWYRQAPGKGRELVAVVRITGSPIYADFVGGRFTISRDNSKNTLYLQMNSLRAEDTAVYYCNTGNFNTYTRRYSPSETWGKGTTVTVSS

SEQ ID NO: 48 - protein, 123 aa, synthetic construct
  REGION 1..123: note = AR8820-2.1
EVQLVESGGGLIQPGGSLRLSCAASGSSISRYAMAWYRQAPGKGRELVSVVRITGSPIYADFVGGRFTISRDNSKNTLYLQMNSLRAEDTAVYYCATGNFNTYTRRYSPSETWGQGTTVTVSS

SEQ ID NO: 49 - protein, 123 aa, synthetic construct
  REGION 1..123: note = AR8820-2.3
EVQLVESGGGLIQPGGSLRLSCAASGSSISRYAMAWYRQAPGKGRELVSVVRITGSPIYADFVGGRFTISRDNSKNTLYLQMNSLRAEDTAVYYCNRGNFNTYTRRYSPSETWGQGTTVTVSS

SEQ ID NO: 50 - protein, 123 aa, synthetic construct
  REGION 1..123: note = AR8820-3.1
EVQLVESGGGLVQPGGSLRLSCAASGSSISRYAMAWYRQAPGKGRELVAVVRITGSPIYADFVGGRFTISRDNAKNSLYLQMNSLRAEDTAVYYCATGNFNTYTRRYSPSETWGQGTLVTVSS

SEQ ID NO: 51 - protein, 123 aa, synthetic construct
  REGION 1..123: note = AR8820-3.2
EVQLVESGGGLVQPGGSLRLSCAASGSSISRYAMAWYRQAPGKGREWVAVVRITGSPIYADFVGGRFTISRDNAKNSLYLQMNSLRAEDTAVYYCATGNFNTYTRRYSPSETWGQGTLVTVSS

SEQ ID NO: 52 - protein, 123 aa, synthetic construct
  REGION 1..123: note = AR8820-3.3
EVQLVESGGGLVQPGGSLRLSCAASGSSISRYAMAWYRQAPGKGRELVAVVRITGSPIYADFVGGRFTISRDNAKNSLYLQMNSLRAEDTAVYYCNTGNFNTYTRRYSPSETWGQGTLVTVSS

SEQ ID NO: 53 - protein, 121 aa, synthetic construct
  REGION 1..121: note = AR8866-1.1
EVQLVESGGGLVQPGGSLRLSCAASGFTFSVWTMSWVRQAPGKGLEWVSDISHSGIYTTYADSVKGRFTISRDNSKNTLYLQMNSLRAEDTAVYYCAKGNRRNSMLSGTLWGQGTLVTVSS

SEQ ID NO: 54 - protein, 121 aa, synthetic construct
  REGION 1..121: note = AR8866-1.2
EVQLVESGGGLVQPGGSLRLSCAASGFTFSVWTMSWVRQAPGKGLEWVSDISHSGIYTTYADSVKGRFTISRDNSKNTLYLQMNSLRAEDTAVYYCAQGNRRNSMLSGTLWGQGTLVTVSS

SEQ ID NO: 55 - protein, 121 aa, synthetic construct
  REGION 1..121: note = AR8866-2.1
EVQLVESGGGLVKPGGSLRLSCAASGFTFSVWTMSWVRQAPGKGLEWVSDISHSGIYTTYADSVKGRFTISRDNAKNSLYLQMNSLRAEDTAVYYCARGNRRNSMLSGTLWGQGTLVTVSS

SEQ ID NO: 56 - protein, 121 aa, synthetic construct
  REGION 1..121: note = AR8866-2.2
EVQLVESGGGLVKPGGSLRLSCAASGFTFSVWTMSWVRQAPGKGLEWVSDISHSGIYTTYADSVKGRFTISRDNAKNSLYLQMNSLRAEDTAVYYCAQGNRRNSMLSGTLWGQGTLVTVSS

SEQ ID NO: 57 - protein, 121 aa, synthetic construct
  REGION 1..121: note = AR8866-3
EVQLVESGGGLVQPGGSLRLSCAASGFTFSVWTMSWVRQAPGKGLVWVSDISHSGIYTTYADSVKGRFTISRDNAKNTLYLQMNSLRAEDTAVYYCAQGNRRNSMLSGTLWGQGTLVTVSS

SEQ ID NO: 58 - protein, 121 aa, synthetic construct
  REGION 1..121: note = AR8866-4.1
EVQLVESGGGLVQPGGSLRLSCAASGFTFSVWTMSWVRQAPGKGLEWVSDISHSGIYTTYADSVKGRFTISRDNAKNSLYLQMNSLRAEDTAVYYCARGNRRNSMLSGTLWGQGTLVTVSS

SEQ ID NO: 59 - protein, 121 aa, synthetic construct
  REGION 1..121: note = AR8866-4.2
EVQLVESGGGLVQPGGSLRLSCAASGFTFSVWTMSWVRQAPGKGLEWVSDISHSGIYTTYADSVKGRFTISRDNAKNSLYLQMNSLRAEDTAVYYCAQGNRRNSMLSGTLWGQGTLVTVSS

SEQ ID NO: 60 - protein, 121 aa, synthetic construct
  REGION 1..121: note = AR8866-5.1
EVQLVESGGGLVQPGGSLRLSCAASGFTFSVWTMSWVRQAPGKGLEWVSDISHSGIYTTYADSVKGRFTISRDNSKNTLYLQMNSLRAEDTAVYYCARGNRRNSMLSGTLWGQGTLVTVSS

SEQ ID NO: 61 - protein, 121 aa, synthetic construct; identical to SEQ ID NO: 54
  REGION 1..121: note = AR8866-5.2

SEQ ID NO: 62 - protein, 121 aa, synthetic construct
  REGION 1..121: note = AR8866-6
QVQLVESGGGVVQPGRSLRLSCAASGFTFSVWTMSWVRQAPGKGLEWVADISHSGIYTTYADSVKGRFTISRDNSKNTLYLQMNSLRAEDTAVYYCAQGNRRNSMLSGTLWGQGTLVTVSS

SEQ ID NO: 63 - protein, 121 aa, synthetic construct
  REGION 1..121: note = AR8875-1.1
EVQLVESGGGLVQPGGSLRLSCAASGFTFSNYAMTWVRQAPGKGLVWVSSIDRGGSITSYADSVKGRFTISRDNAKNTLYLQMNSLRAEDTAVYYCARADSTYSRLTAAYWGQGTLVTVSS

SEQ ID NO: 64 - protein, 12 aa, synthetic construct
  REGION 1..12: note = CDR3 of AR8775-1.1
ADSTYSRLTAAY

SEQ ID NO: 65 - protein, 121 aa, synthetic construct
  REGION 1..121: note = AR8875-1.2
EVQLVESGGGLVQPGGSLRLSCAASGFTFSNYAMTWVRQAPGKGLVWVSSIDRGGSITSYADSVKGRFTISRDNAKNTLYLQMNSLRAEDTAVYYCARSDSTYSRLTAAYWGQGTLVTVSS

SEQ ID NO: 66 - protein, 12 aa, synthetic construct
  REGION 1..12: note = CDR3 of AR8775-1.2
SDSTYSRLTAAY

SEQ ID NO: 67 - protein, 121 aa, synthetic construct
  REGION 1..121: note = AR8875-2
EVQLLESGGGLVQPGGSLRLSCAASGFTFSNYAMTWVRQAPGKGLEWVSSIDRGGSITSYADSVKGRFTISRDNSKNTLYLQMNSLRAEDTAVYYCARADSTYSRLTAAYWGQGTLVTVSS

SEQ ID NO: 68 - protein, 121 aa, synthetic construct
  REGION 1..121: note = AR8875-3.2
EVQLVESGGGLVKPGGSLRLSCAASGFTFSNYAMTWVRQAPGKGLEWVSSIDRGGSITSYADSVKGRFTISRDNAKNSLYLQMNSLRAEDTAVYYCARSDSTYSRLTAAYWGQGTLVTVSS

SEQ ID NO: 69 - protein, 121 aa, synthetic construct
  REGION 1..121: note = AR8875-4.1
EVQLVESGGGLVQPGGSLRLSCAASGFTFSNYAMTWVRQAPGKGLEWVSSIDRGGSITSYADSVKGRFTISRDNAKNSLYLQMNSLRAEDTAVYYCARADSTYSRLTAAYWGQGTLVTVSS

SEQ ID NO: 70 - protein, 121 aa, synthetic construct
  REGION 1..121: note = AR8875-4.2
EVQLVESGGGLVQPGGSLRLSCAASGFTFSNYAMTWVRQAPGKGLEWVSSIDRGGSITSYADSVKGRFTISRDNAKNSLYLQMNSLRAEDTAVYYCARSDSTYSRLTAAYWGQGTLVTVSS

SEQ ID NO: 71 - protein, 121 aa, synthetic construct
  REGION 1..121: note = AR8875-5
QVQLVESGGGVVQPGRSLRLSCAASGFTFSNYAMTWVRQAPGKGLEWVASIDRGGSITSYADSVKGRFTISRDNSKNTLYLQMNSLRAEDTAVYYCARADSTYSRLTAAYWGQGTLVTVSS

SEQ ID NO: 72 - protein, 121 aa, synthetic construct
  REGION 1..121: note = AR8875-6
EVQLVESGGGLVQPGRSLRLSCAASGFTFSNYAMTWVRQAPGKGLEWVSSIDRGGSITSYADSVKGRFTISRDNAKNSLYLQMNSLRAEDTALYYCARADSTYSRLTAAYWGQGTLVTVSS

SEQ ID NO: 73 - protein, 229 aa, synthetic construct
  REGION 1..229: note = Human IgG4 Fc
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 74 - protein, 227 aa, synthetic construct
  REGION 1..227: note = Mouse IgG1 Fc
VPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

(19) United States
(12) Patent Application Publication
(10) Pub. No.: US 2024/0352128 A1
(43) Pub. Date: Oct. 24, 2024

(54) ANTIBODIES AND VARIANTS THEREOF AGAINST HUMAN CD16a

(71) Applicant: Nanjing GenScript Biotech Co., Ltd., Nanjing (CN)

(72) Inventors: Gang Liu, Nanjing (CN); Zhongdao Li, Nanjing (CN); Zhihui Zhao, Nanjing (CN); Cuiying Shao, Nanjing (CN); Bin Liu, Nanjing (CN); Yi Jin, Nanjing (CN); Liusong Yin, Nanjing (CN)

(73) Assignee: Nanjing GenScript Biotech Co., Ltd., Nanjing, JS (CN)

(21) Appl. No.: 18/682,827

(22) PCT Filed: Aug. 25, 2022
(86) PCT No.: PCT/CN2022/114642

§ 371 Date: Feb. 9, 2024

(30) Foreign Application Priority Data
Aug. 25, 2021 (WO) PCT/CN2021/114549

(51) Int. Cl. C07K 16/28; A61K 39/00; A61P 35/00; A61P 37/04

(52) U.S. Cl. CPC C07K 16/283; A61P 35/00; A61P 37/04; A61K 2039/505; C07K 2317/24; C07K 2317/33; C07K 2317/52; C07K 2317/565; C07K 2317/569

(57) ABSTRACT

Binding molecules are provided. The binding molecules specifically bind to CD16a (FcγRIIIA) expressed on the surface of natural killer (NK) cells, but do not bind to CD16b (FcγRIIIB). The use of such binding molecules in the stimulation and activation of primary human NK cells is also disclosed.

### CROSS REFERENCE TO RELATED APPLICATIONS

This is the U.S. National Stage of International Application No. PCT/CN2022/114642, filed Aug. 25, 2022, which was published in English under PCT Article 21(2), herein incorporated by reference in its entirety, which in turn claims the benefit of International Application No. PCT/CN2021/114549, filed Aug. 25, 2021.

### REFERENCE TO AN ELECTRONIC SEQUENCE LISTING

The Sequence Listing is submitted as an ST.26 file named 9916-111481-01_ST26.xml, 98,445 bytes, dated Feb. 9, 2024, which is incorporated by reference herein.

### BACKGROUND

Fcγ receptors (FcγR) are cell surface receptors that bind the Fc region of immunoglobulin G (IgG). These receptors form antibody-antigen complexes and play a part in effector cell responses. For example, cross-linking of activating Fcγ receptors by immune complexes can result in the phagocytosis of pathogens, killing of foreign and transformed cells by-direct cytotoxicity, the clearance of toxic substances, and the initiation of an inflammatory response. In antibody therapies, IgG recognizes targets through the antigen binding fragments and binds multiple types of FcγRs expressed on leukocytes with the Fc domain to elicit a cell-mediated response to treat leukemia, lymphomas, tumors, autoimmune disease and other diseases.

There are three classes of Fcγ receptors, FcγRI, FcγRII, and FcγRIII. FcγRIII, also called CD16 is a low affinity receptor for the Fc portion of some IgGs known to be involved in antibody-dependent cellular cytotoxicity (ADCC), is the best characterized membrane receptor responsible for triggering of target cell lysis by NK cells. Human FcγRIII exists as two isoforms, FcγRIIIA (CD16a) and FcγRIIIB (CD16b), that share 96% sequence identity in their extracellular immunoglobulin-binding regions. FcγRIIIA (CD16a) is expressed on macrophages, mast cells, and NK cells as a transmembrane receptor. FcγRIIIB (CD16b), however, is present on polymorphonuclear granulocytes (PMN) as a glycosyl-phosphatidylinositol (GPI)-anchored receptor (FcγRIIIB isoform), which cannot trigger tumor cell killing. In addition, FcγRIIIB exists as a soluble receptor in serum and upon binding to antibodies may trigger side-effects via the formation of immune complexes in vivo.

FcγR-expressing effector cells have been shown to be involved in destroying tumor cells via ADCC. This has led to the development of several immunotherapeutic approaches to cancer therapy, which involve the use of FcγR activities. As cellular mediators of innate immunity, NK cells are professional cell killers and, unlike T cells, tend to exist in constitutively activated states not requiring additional (pre-) stimulation. The majority of anti-CD16 antibodies that have been produced to date, are unable to distinguish between the two CD16 isoforms, FcγRIIIA and FcγRIIIB.

There is a need for anti-CD16a binding molecules or antibodies specific to CD16a as against CD16b and have desirable therapeutic profiles.

### SUMMARY OF THE INVENTION

In one aspect, the present disclosure provides an isolated antibody, or an antigen-binding portion thereof, comprising: a monomeric variable domain comprising a CDR1region, a CDR2 region, and a CDR3 region comprising the amino acid sequences of: SEQ ID NOs: 2, 3, and 4, respectively; or SEQ ID NOs: 6, 7, and 8, respectively; or SEQ ID NOs: 14, 15, and 16, respectively; or SEQ ID NOs: 14, 15, and 64, respectively; or SEQ ID NOs: 14, 15, and 66, respectively; or SEQ ID NOs: 18, 19, and 20, respectively; or SEQ ID NOs: 26, 27, and 28, respectively; or a variant thereof comprising up to about 3 amino acid substitutions in any one or more of the CDR1 region, the CDR2 region, and the CDR3 region.

In some embodiments, the CDR1 region, the CDR2 region, and the CDR3 region comprises the amino acid sequences of: SEQ ID NOs: 2, 3, and 4, respectively; or SEQ ID NOs: 6, 7, and 8, respectively; or SEQ ID NOs: 18, 19, and 20, respectively; or SEQ ID NOs: 26, 27, and 28, respectively; or a variant thereof comprising up to about 3 amino acid substitutions in any one or more of the CDR1 region, the CDR2 region, and the CDR3 region.

In some embodiments, the antibody or antigen-binding portion thereof specifically binds to human CD16a.

In some embodiments, the antibody or antigen-binding portion thereof comprises or is a single domain antibody (sdAb) or a V_{H}H domain.

In some embodiments, the antibody or antigen-binding portion thereof is camelid, chimeric, human or humanized.

In some embodiments, the monomeric variable domain comprises an amino acid sequence having at least 80%, 85%, 90%, 92%, 94%, 95%, 96%, 97%, 98%, or 99% identity to the amino acid sequence selected from the group consisting of SEQ ID NOs: 1, 5, 13, 17, 25, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, and 62. In certain embodiments, the monomeric variable domain comprises an amino acid sequence selected from the group consisting of SEQ ID NOs: 1, 5, 13, 17, 25, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, and 62.

In some embodiments, the monomeric variable domain comprises an amino acid sequence having at least 80%, 85%, 90%, 92%, 94%, 95%, 96%, 97%, 98%, or 99% identity to the amino acid sequence selected from the group consisting of SEQ ID NOs: 29, 30, 31, 32, 33, 34, 35, 36, 37, 38, 39, 40, 41, 42, 43, 44, 45, 46, 47, 48, 49, 50, 51, and 52. In certain embodiments, the monomeric variable domain comprises an amino acid sequence selected from the group consisting of SEQ ID NOs: 29, 30, 31, 32, 33, 34, 35, 36, 37, 38, 39, 40, 41, 42, 43, 44, 45, 46, 47, 48, 49, 50, 51, and 52.

In some embodiments, the monomeric variable domain comprises an amino acid sequence having at least 80%, 85%, 90%, 92%, 94%, 95%, 96%, 97%, 98%, or 99% identity to the amino acid sequence selected from the group consisting of SEQ ID NOs: 42, 44, 45, 47, and 50. In certain of these embodiments, the monomeric variable domain comprises an amino acid sequence selected from the group consisting of SEQ ID NOs: 42, 44, 45, 47, and 50.

In another aspect, the present disclosure provides an isolated antibody, or an antigen-binding portion thereof, comprising a monomeric variable domain comprising an amino acid sequence selected from the group consisting of SEQ ID NOs: 1, 5, 13, 17, 25, 29, 30, 31, 32, 33, 34, 35, 36, 37, 38, 39, 40, 41, 42, 43, 44, 45, 46, 47, 48, 49, 50, 51, 5253, 54, 55, 56, 57, 58, 59, 60, 61, and 62. The antibody can be a humanized antibody, and the monomeric variable domain can comprise the amino acid sequence selected from the group consisting of SEQ ID NOs: 42, 44, 45, 47, and 50.

In any of the isolated antibody, or antigen-binding portion thereof disclosed herein, the antibody or the antigen-binding portion thereof can further comprise an IgG Fc region fused with the monomeric variable domain. The IgG Fc region can be a human IgG4 Fc region or a mouse IgG1 Fc region. In a certain embodiment, the human IgG4 Fc region may comprise an amino acid sequence of SEQ ID NO:73. In a certain embodiment, the mouse IgG1 Fc region may comprise an amino acid sequence of SEQ ID NO:74.

In a further aspect, the present disclosure provides a bispecific molecule, an immunoconjugate, or a chimeric antigen receptor, comprising the antibody or antigen-binding portion thereof as described herein.

In a further aspect, the present disclosure provides a nucleic acid molecule encoding the antibody or antigen-binding portion thereof, as described herein, or the bispecific molecule, an immunoconjugate, or a chimeric antigen receptor thereof. In a further aspect, the present disclosure provides an expression vector containing the nucleic acid molecule. In a further aspect, the present disclosure provides a host cell containing the expression vector.

In a further aspect, the present disclosure provides a pharmaceutical composition comprising the antibody or antigen-binding portion thereof as described herein, or the bispecific molecule, an immunoconjugate, or a chimeric antigen receptor as described herein, and a pharmaceutically acceptable carrier. In some embodiments, the pharmaceutical can further comprise a cytotoxic agent. In a further aspect, the present disclosure provides a method for treating a cancer disease in a subject, comprising administering to the subject a therapeutically effective amount of the pharmaceutical composition as described herein. The cancer disease is selected from the group consisting of adrenal cancer, bladder cancer, breast cancer, colorectal cancer, gastric cancer, glioblastoma, kidney cancer, non-small-cell lung cancer, acute lymphocytic leukemia, acute myeloid leukemia, chronic lymphocytic leukemia, chronic myeloid leukemia, hairy cell leukemia, Burkett's lymphoma, diffuse large B cell lymphoma, follicular lymphoma, mantle cell lymphoma, marginal zone lymphoma, non-Hodgkin's lymphoma, small lymphocytic lymphoma, multiple myeloma, melanoma, ovarian cancer, pancreatic cancer, prostate cancer, skin cancer, renal cell carcinoma, testicular cancer, and uterine cancer.

In a further aspect, the present disclosure provides a method of promoting NK cell proliferation in a subject, comprising administering to the subject a therapeutically effective amount of the pharmaceutical composition as described herein.

### BRIEF DESCRIPTION OF THE DRAWINGS

FIGS. 1A and 1B show FACS binding curves of certain embodiments of antibodies the present disclosure with human CD16a proteins.

FIGS. 2A and 2B show FACS binding curves of certain embodiments of antibodies the present disclosure with human CD16b proteins.

FIG. 2C shows FACS binding curves of certain embodiments of antibodies the present disclosure with human CD16a proteins.

FIG. 2D shows FACS binding affinities certain embodiments of antibodies the present disclosure with human CD16b proteins.

FIG. 3 shows cytotoxicity assay results of certain embodiments of antibodies the present disclosure in triggering NK cells' cytotoxicity.

FIGS. 4A and 4B show binding affinities of certain humanized sdAbs of the present disclosure with human CD16a proteins.

FIGS. 5A and 5B show binding affinities of certain humanized sdAbs of the present disclosure with human CD16b proteins.

FIG. 6A shows FACS binding curves and data of certain humanized sdAbs of the present disclosure with human CD16a proteins; FIG. 6B shows FACS binding curves and data of these humanized sdAbs with human CD16b proteins.

FIG. 7A shows FACS binding curves and data of certain humanized sdAbs of the present disclosure with human CD16a proteins; FIG. 7B shows FACS binding curves and data of these humanized sdAbs with human CD16b proteins.

FIG. 8A shows FACS binding curves and data of certain humanized sdAbs of the present disclosure with human CD16a proteins; FIG. 8B shows FACS binding curves and data of these humanized sdAbs with human CD16b proteins.

FIG. 9A shows FACS binding curves and data of certain humanized sdAbs of the present disclosure with human CD16a proteins; FIG. 9B shows FACS binding curves and data of these humanized sdAbs with human CD16b proteins.

FIG. 10A shows FACS binding curves and data of certain humanized sdAbs of the present disclosure with human CD16a proteins; FIG. 10B shows FACS binding curves and data of these humanized sdAbs with human CD16b proteins.

FIGS. 11A and 11B show lactate dehydrogenase (LDH) cytotoxicity assay results with NK92/CD16a-158V/V cells of certain humanized sdAbs of the present disclosure with human CD16a proteins.

FIG. 12 shows lactate dehydrogenase (LDH) cytotoxicity assay results with NK92/CD16a-158V/V cells of certain humanized sdAbs of the present disclosure with human CD16a proteins by dose dependent response of reverse ADCC bioassay.

FIGS. 13A and 13B show FACS Cyno binding curves and data of certain humanized sdAbs of the present disclosure with human CD16a proteins.

### DETAILED DESCRIPTION

In one aspect, the present disclosure provides anti-CD16 llama single domain antibodies that exhibit specificity for FcγRIIIA (CD16a) but do not bind with specificity to FcγRIIIB (CD16b). The present disclosure further provides humanized sdAbs based on these llama sdAbs and fusion proteins thereof. The antibodies (or binding molecules) of the present disclosure can activate NK cells upon binding, and can be used as a component of a bispecific or multispecific binding molecule directed against disease-associated cells.

The term “antigen-binding portion” of an antibody as used herein refers to one or more fragments of an antibody that retain the ability to bind to an antigen (e.g., a CD16a protein). This can include a heavy chain variable region containing a single variable domain, as well as a bigger molecule that includes such a domain and another polypeptide segment chemically linked to it. Such single chain antibodies are also intended to be encompassed within the term “antigen-binding portion”.

An “isolated antibody” as used herein refers to an antibody that is substantially free of other antibodies having different antigenic specificities. An isolated antibody that specifically binds a CD16a protein is substantially free of antibodies that do not bind to a CD16a protein. An isolated antibody that binds a human CD16a may also bind other antigens, such as human CD16b protein or CD16a proteins from other species. An isolated antibody can also be substantially free of other cellular material and/or chemicals.

As used herein, the terms “CD16a binding protein,” “CD16a antibody,” and “anti-CD16a antibody,” are used interchangeably and refer to a variety of molecules that bind CD16a via an interaction with VL and/or VH domains (as distinct from Fc-mediated binding). Examples of CD16a binding proteins includes chimeric, humanized and human antibodies (e.g., comprising 2 heavy and 2 light chains), fragments thereof (e.g., Fab, Fab′, F(ab′)2, and Fv fragments), bifunctional or multifunctional antibodies, single chain antibodies, fusion proteins (e.g., phage display fusion proteins), “minibodies” (see, e.g., U.S. Pat. No. 5,837,821) and other antigen binding proteins comprising a VL and/or VH domain or fragment thereof.

The terms “monoclonal antibody” as used herein refer to a preparation of antibody molecules of single molecular composition.

The term “single domain antibody” or “sdAb” refers to a single antigen-binding polypeptide comprising a single monomeric variable antibody domain having three complementary determining regions (CDRs), which is capable of binding to an antigen without pairing with a corresponding CDR-containing polypeptide. In some cases, the single domain antibody is engineered from a camelid heavy chain antibodies (HCAb), and is also called the V_{H}H domain or fragment of the HCAb. The single domain antibody is a kind of antigen-binding portion of a heavy chain only antibody. The V_{H}Hs may also be known as Nanobodies. Camelid sdAb is one of the smallest known antigen binding antibody fragments (see, e.g., Hamers-Casterman et al., Nature 363:446-8 (1993); Greenberg et al., Nature 374:168-73 (1995); Hassanzadeh-Ghassabeh et al., Nanomedicine (Lond), 8:1013-26 (2013)). As examples, particular single domain antibodies in the Examples of the present disclosure are referred to AR[digits and/or other symbols], e.g., AR8866, AR8866-1.1, AR8804, AR8804-1.1, etc.

As used herein, an antibody or molecule that “specifically binds to human CD16a” refers to an antibody or polypeptide molecule that binds to human CD16a protein but does not substantially bind to proteins that are not human CD16a proteins. In particular, an antibody or molecule referred to herein as specifically binding to human CD16a should have a binding affinity against human CD16a of at least 100-fold higher than its binding affinity against human CD16b.

As used herein, the term “fused protein”, “fusion protein”, “fusion antibody”, or “fused antibody” refers to an antibody molecule that include both a single domain antibody (e.g., V_{H}H) part and an IgG constant region Fc (such as human IgG4 Fc region or mouse IgG1 Fc region), where the two parts are chemically linked, e.g., by recombinant methods. For example, sdAbs can be directly fused to the IgG Fc region with the native hinge region of the IgG. The sdAb is located upstream, i.e., closer to the N-terminus, of the IgG Fc in the fused antibody, where the sdAb and the IgG Fc region are intervened by the hinge region. A fused antibody protein can be referenced herein as its constituent parts, e.g., AR8776-sdAb-hIgG4Fc refers to a fused protein or antibody made up by AR8776-sdAb fused with the human IgG4 Fc region.

The term “camelid antibody”, as used herein, is intended to include antibodies having variable regions (or more specifically the single domain antibodies or V_{H}H fragments) in which both the framework and CDR regions are derived from camelid germline heavy chain only antibody sequences. Furthermore, if the antibody contains a constant region, the constant region can also be derived from camelid germline antibody sequences. The camelid antibodies of the invention can include amino acid residues not encoded by camelid germline antibody sequences (e.g., mutations introduced by random or site-specific mutagenesis in vitro or by somatic mutation in vivo). However, the term “camelid antibody”, as used herein, is not intended to include antibodies in which CDR sequences derived from the germline of another mammalian species have been grafted onto camelid framework sequences.

The term “chimeric antibody” refers to an antibody made by combining genetic material from a nonhuman (e.g., camelid) source with genetic material from a human being. Or more generally, a chimeric antibody is an antibody having different parts produced from genetic material of different species.

The term “humanized antibody”, as used herein, refers to an antibody from non-human (e.g., camelid) species whose protein sequences have been modified to increase similarity to antibody variants produced naturally in humans.

The terms percent “identity” as used herein in the context of two or more nucleic acids or proteins/peptides, refer to two or more sequences or subsequences that have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software that can be used to obtain alignments of amino acid or nucleotide sequences are well-known in the art. These include, but are not limited to, BLAST, ALIGN, Megalign, BestFit, GCG Wisconsin Package, and variants thereof.

As used herein, the term “subject” includes any human or nonhuman animal. The term “nonhuman animal” includes all vertebrates, e.g., mammals and non-mammals. In certain embodiments, the subject is a human.

The CDRs of an antibody are defined by those skilled in the art using a variety of methods/systems. These systems and/or definitions have been developed and refined over a number of years and include Kabat, Chothia, IMGT, AbM, and Contact. The Kabat definition is based on sequence variability and is commonly used. The Chothia definition is based on the location of the structural loop regions. The IMGT system is based on sequence variability and location within the structure of the variable domain. The AbM definition is a compromise between Kabat and Chothia. The Contact definition is based on analyses of the available antibody crystal structures. An Exemplary system is a combination of Kabat and Chothia.

Protein and nucleic acid sequences for CD16a are reported in Genbank as accession numbers P08637 (protein) and X52645 (nucleic acid) and in SWISS-PROT as accession number CAA36870. Protein and nucleic acid sequences for CD16b are reported in Genbank as accession numbers O75015 (protein) and X16863 (nucleic acid) and in SWISS-PROT as CAA34753.

### Antibody Targeting Human CD16a (or Anti-Human CD16a Antibody)

In one aspect, the present disclosure provides an isolated antibody, or an antigen-binding portion thereof, comprising a monomeric variable domain comprising a CDR1region, a CDR2 region, and a CDR3 region comprising the amino acid sequences of: SEQ ID NOs: 2, 3, and 4, respectively; or SEQ ID NOs: 6, 7, and 8, respectively; or SEQ ID NOs: 14, 15, and 16, respectively; or SEQ ID NOs: 14, 15, and 64, respectively; or SEQ ID NOs: 14, 15, and 66, respectively; or SEQ ID NOs: 18, 19, and 20, respectively; or SEQ ID NOs: 26, 27, and 28, respectively; or a variant thereof comprising up to about 3 amino acid substitutions in any one or more of the CDR1 region, the CDR2 region, and the CDR3 region. In some embodiments, the CDR1 region, the CDR2 region, and the CDR3 region comprises the amino acid sequences of: SEQ ID NOs: 2, 3, and 4, respectively; or SEQ ID NOs: 6, 7, and 8, respectively; or SEQ ID NOs: 18, 19, and 20, respectively; or SEQ ID NOs: 26, 27, and 28, respectively; or a variant thereof comprising up to about 3 amino acid substitutions in any one or more of the CDR1 region, the CDR2 region, and the CDR3 region.

In some embodiments, the antibody or antigen-binding portion thereof specifically binds to human CD16a.

In some embodiments, the antibody or antigen-binding portion thereof comprises or is a single domain antibody (sdAb) or a V_{H}H domain.

In some embodiments, the antibody or antigen-binding portion thereof is camelid, chimeric, human or humanized.

In some embodiments, the monomeric variable domain comprises an amino acid sequence having at least 80%, 85%, 90%, 92%, 94%, 95%, 96%, 97%, 98%, or 99% identity to the amino acid sequence selected from the group consisting of SEQ ID NOs: 1, 5, 13, 17, 25, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, and 62. In certain embodiments, the monomeric variable domain comprises an amino acid sequence selected from the group consisting of SEQ ID NOs: 1, 5, 13, 17, 25, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, and 62.

In some embodiments, the monomeric variable domain comprises an amino acid sequence having at least 80%, 85%, 90%, 92%, 94%, 95%, 96%, 97%, 98%, or 99% identity to the amino acid sequence selected from the group consisting of SEQ ID NOs: 29, 30, 31, 32, 33, 34, 35, 36, 37, 38, 39, 40, 41, 42, 43, 44, 45, 46, 47, 48, 49, 50, 51, and 52. In certain embodiments, the monomeric variable domain comprises an amino acid sequence selected from the group consisting of SEQ ID NOs: 29, 30, 31, 32, 33, 34, 35, 36, 37, 38, 39, 40, 41, 42, 43, 44, 45, 46, 47, 48, 49, 50, 51, and 52.

In some embodiments, the monomeric variable domain comprises an amino acid sequence having at least 80%, 85%, 90%, 92%, 94%, 95%, 96%, 97%, 98%, or 99% identity to the amino acid sequence selected from the group consisting of SEQ ID NOs: 42, 44, 45, 47, and 50. In certain of these embodiments, the monomeric variable domain comprises an amino acid sequence selected from the group consisting of SEQ ID NOs: 42, 44, 45, 47, and 50.

In another aspect, the present disclosure provides an isolated antibody, or an antigen-binding portion thereof, comprising a monomeric variable domain comprising an amino acid sequence selected from the group consisting of SEQ ID NOs: 1, 5, 13, 17, 25, 29, 30, 31, 32, 33, 34, 35, 36, 37, 38, 39, 40, 41, 42, 43, 44, 45, 46, 47, 48, 49, 50, 51, 5253, 54, 55, 56, 57, 58, 59, 60, 61, and 62. The antibody can be a humanized antibody, and the monomeric variable domain can comprise the amino acid sequence selected from the group consisting of SEQ ID NOs: 42, 44, 45, 47, and 50.

The antibody or antigen-binding portion thereof described herein can further comprise an IgG Fc region fused with the monomeric variable domain. For example, the IgG Fc region can be a human IgG4 Fc region or a mouse IgG1 Fc region. In a certain embodiment, the human IgG4 Fc region may comprise an amino acid sequence of SEQ ID NO:73. In a certain embodiment, the mouse IgG1 Fc region may comprise an amino acid sequence of SEQ ID NO:74.

In a further aspect, the present disclosure provides a bispecific molecule, an immunoconjugate (or antibody drug conjugate), or a chimeric antigen receptor, comprising the antibody or antigen-binding portion thereof as described herein.

The term “bispecific molecule” refers to an antibody of the present disclosure linked to at least one other functional molecule, e.g., another peptide or protein (e.g., another antibody or ligand for a receptor). A bispecific molecule can have at least two different binding sites or target molecules, and can includes molecules that have three or more specificities.

The term “immunoconjugate” refers to an antibody of the present disclosure conjugated to a therapeutic agent, such as cytotoxins, alkylating agents, DNA minor groove binders, DNA intercalators, DNA crosslinkers, histone deacetylase inhibitors, nuclear export inhibitors, proteasome inhibitors, topoisomerase I or II inhibitors, etc.. In the ADC, the antibody and therapeutic agent can be are conjugated via a cleavable linker such as a peptidyl, disulfide,

The term “chimeric antigen receptor” or “CAR” refers to an engineered receptor that grafts a defined specificity onto an immune effector cell, typically a T cell, and augments T-cell function. The new generation CAR comprises an extracellular binding domain comprising a single domain antibody, a hinge region, a transmembrane domain, and an intracellular signaling domain (mainly CD3-zeta's cytoplasmic domain, which is the primary transmitter of T cell activation signals, plus one or more co-stimulatory domains). The CARs may further have factors that enhance T cell expansion, persistence, and anti-tumor activity, such as cytokines and co-stimulatory ligands.

In yet a further aspect, the present disclosure provides a nucleic acid molecule encoding the antibody or antigen-binding portion thereof of any of the antibody (or antigen-binding portion thereof), or the bispecific molecule, an immunoconjugate, or a chimeric antigen receptor of described herein. A host cell (e.g., a CHO cell, or a lymphocytic cell, or microorganisms, such as E. coli, and fungi, such as yeast) containing an expression vector containing the nucleic acid molecule, can be used to produce antibodies of the present disclosure, preferably single domain antibodies.

Methods of preparing single domain antibodies have been described. See, for example, Els Pardon et al, Nature Protocol, 2014; 9(3): 674. The commonly used method for single domain antibody preparation involves immunization of a Camelid species with the antigen of interest such as CD16a, recovery of lymphocytes from the immunized animal, preparation of the cDNAs, generation of a phage display library using standard cloning protocols, and three to four rounds of phage screening to enrich antigen-specific binders. A diverse single domain antibody library can also be prepared by introducing diversity into a V_{H}H scaffold synthetically.

The single domain antibodies can also be isolated from phage display libraries expressing camelid single domain antibodies. Screening of phage libraries can be accomplished by various techniques known in the art. For example, a camelid naïve single domain antibody library may be screened for antibodies binding to the coronavirus spike protein by solution panning with colorimetric plates coated with the receptor binding domain of CD16a protein over several rounds of selection with increasing stringency. Isolates may be first expressed as single domain antibodies and screened for binding to the receptor binding domain by ELISA, and the selected isolates may then be cloned and expressed as single domain antibody linked to IgG4 or IgG1 Fc region, reanalyzed for binding to CD16a protein by ELISA and/or SPR and for functional activity and transfected in a CHO mammalian cell line for expression of the single domain antibodies.

In one embodiment, DNA encoding single domain antibody or antigen-binding portion thereof of the present disclosure can be obtained by standard molecular biology techniques is inserted into one or more expression vectors such that the genes are operatively linked to transcriptional and translational regulatory sequences. The term “operatively linked” is intended to mean that an antibody gene is ligated into a vector such that transcriptional and translational control sequences within the vector serve their intended function of regulating the transcription and translation of the antibody gene. The term “regulatory sequence” is intended to include promoters, enhancers and other expression control elements (e.g., polyadenylation signals) that control the transcription or translation of the antibody genes. Such regulatory sequences are described, e.g., in Goeddel (Gene Expression Technology. Methods in Enzymology 185, Academic Press, San Diego, Calif. (1990)). Preferred regulatory sequences for mammalian host cell expression include viral elements that direct high levels of protein expression in mammalian cells, such as promoters and/or enhancers derived from cytomegalovirus (CMV), Simian Virus 40 (SV40), adenovirus, e.g., the adenovirus major late promoter (AdMLP) and polyoma. Alternatively, nonviral regulatory sequences can be used, such as the ubiquitin promoter or β-globin promoter. Still further, regulatory elements composed of sequences from different sources, such as the SRα promoter system, which contains sequences from the SV40 early promoter and the long terminal repeat of human T cell leukemia virus type 1 (Takebe et al., (1988) Mol. Cell. Biol. 8:466-472). The expression vector and expression control sequences are chosen to be compatible with the expression host cell used.

The antibody encoding DNA can be inserted into the expression vector. The recombinant expression vector can encode a signal peptide that facilitates secretion of the antibody chain from a host cell. The antibody encoding DNA can be cloned into the vector such that the signal peptide is linked in-frame to the amino terminus of the antibody encoding DNA. The signal peptide can be an immunoglobulin signal peptide or a heterologous signal peptide (i.e., a signal peptide from a non-immunoglobulin protein).

For expression of sdAbs (or the sdAb-IgGFc), the expression vector(s) encoding the antibody chain can be transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Mammalian host cells for expressing the antibodies of the present disclosure include HEK293 cells, Chinese Hamster Ovary (CHO cells), NSO myeloma cells, COS cells and SP2 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

### Pharmaceutical Compositions and Methods of Treatment

In another aspect, the present disclosure provides a pharmaceutical composition comprising one or more antibodies of the present invention, or the bispecific molecule, an immunoconjugate, or a chimeric antigen receptor described herein, together with a pharmaceutically acceptable carrier in accordance with conventional techniques.

The composition may comprise one or more additional pharmaceutically active ingredients, such as another antibody, a drug, e.g., a cytotoxic or anti-tumor agent. The pharmaceutical compositions of the invention also can be administered in a combination therapy with, for example, another anti-cancer agent, another anti-inflammatory agent, etc. As used herein, “pharmaceutically acceptable carrier” includes pharmaceutically acceptable carriers, excipients or stabilizers. These include but are not limited solvents, dispersion media, coatings, antibacterial and antifungal agents, isotonic and absorption delaying agents, surface active agents, thickening or emulsifying agents, solid binders, dispersion or suspension aids, solubilizers, colorants, flavoring agents, coatings, disintegrating agents, lubricants, sweeteners, preservatives, isotonic agents, and the like that are physiologically compatible. The selection of suitable carrier is within the knowledge of an artisan skilled in the art.

The pharmaceutical composition can be suitable for intravenous, intramuscular, subcutaneous, parenteral, epidermal, and other routes of administration. Depending on the route of administration, the active ingredient can be coated with a material or otherwise loaded in a material or structure to protect it from the action of acids and other natural conditions that may inactivate it. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, an antibody of the invention can be administered via a non-parenteral route, such as a topical, epidermal or mucosal route of administration, e.g., intranasally, orally, vaginally, rectally, sublingually or topically.

In another aspect, the present disclosure provides a method for treating an immune-mediated disease such as cancer in a subject, e.g., a human or non-human mammal, comprising administering to the subject a therapeutically effective amount of the pharmaceutical composition described herein. The cancer can be selected from the group consisting of adrenal cancer, bladder cancer, breast cancer, colorectal cancer, gastric cancer, glioblastoma, kidney cancer, non-small-cell lung cancer, acute lymphocytic leukemia, acute myeloid leukemia, chronic lymphocytic leukemia, chronic myeloid leukemia, hairy cell leukemia, Burkett's lymphoma, diffuse large B cell lymphoma, follicular lymphoma, mantle cell lymphoma, marginal zone lymphoma, non-Hodgkin's lymphoma, small lymphocytic lymphoma, multiple myeloma, melanoma, ovarian cancer, pancreatic cancer, prostate cancer, skin cancer, renal cell carcinoma, testicular cancer, and uterine cancer.

In another aspect, the present disclosure provides a method of promoting NK cell proliferation in a subject, comprising administering to the subject a therapeutically effective amount of the pharmaceutical composition described herein.

In the administration of the composition to the subject, dosage regimens can be adjusted to provide the optimum desired response (e.g., a therapeutic response). Single bolus or divided doses can be administered based on the subject, the disease to be treated, etc. Dosage unit form as used herein refers to physically discrete units suited as unitary dosages for the subjects to be treated. Each unit contains a predetermined quantity of active ingredient calculated to produce the desired therapeutic effect in association with the required pharmaceutical carrier. Sustained release formulation can be used in which case less frequent administration is required.

For administration of an antibody of the present disclosure, the dosage may range from about 0.0001 to 100 mg/kg, and more usually 0.01 to 5 mg/kg, of the body weight of the subject. For example, dosages can be 0.3 mg/kg body weight, 1 mg/kg body weight, 3 mg/kg body weight, 5 mg/kg body weight or 10 mg/kg body weight or within the range of 1-10 mg/kg. A suitable treatment regime can be once per week, once every two weeks, once every three weeks, once every four weeks, once a month, etc. Example dosage regimens for an anti-CD16a antibody of the invention can include 1 mg/kg body weight or 3 mg/kg body weight via intravenous administration.

A “therapeutically effective amount” or “therapeutically effective amount” of an antibody targeting CD16a of the invention preferably results in a decrease in severity of disease symptoms, an increase in frequency and/or duration of disease symptom-free periods, prevention or reduction of likelihood of impairment or disability due to the disease affliction, or inhibition or delaying of the progression of disease. For example, for the treatment of tumor-bearing subjects, a “therapeutically effective amount” of an antibody composition may inhibits tumor growth by at least about 20%, more preferably by at least about 40%, even more preferably by at least about 60%, and still more preferably by at least about 80% relative to untreated subjects.

### EXAMPLES

The examples below are intended to be purely exemplary of the invention and should therefore not be considered to limit the invention in any way. The following examples and detailed description are offered by way of illustration and not by way of limitation.

### Example 1: Generation of Anti-CD16a Llama Single Domain Antibodies (sdAbs)

### Immunization

The llama was immunized with recombinant human CD16a-His proteins (Acro Biosystems; catalog #: CDA-H52S1) under current animal welfare regulations. For immunization, the antigen was administrated in PBS solution or formulated as an emulsion with CFA (Complete Freund's adjuvant; primary immunization) or IFA (incomplete Freund's adjuvant; boost immunizations). The antigen was administered subcutaneously at the neck. The animal received 6 injections of the emulsion, including the primary immunization with 200 μg of antigen in CFA emulsion, the subsequent 3 boosts with 100 μg of antigen in IFA emulsion at one-week interval and the following 2 injections with 50 μg of antigen in IFA emulsion at one-week interval. After multiple rounds of immunization, a blood sample of 100 ml was collected. Peripheral blood lymphocytes (PBLs), as the genetic source of the llama heavy chain antibodies (HCAbs) were isolated from the blood sample by gradient centrifugation. Variable fragments targeting CD16a from AFM13 (U.S. Pat. No. 9,035,026 B2, Affirmed GMBH, Germany) was incorporated into either human IgG4 or mouse IgG1 format as positive control (and reference antibody), and normal human IgG4 or mouse IgG1 was set as isotype control, respectively.

### Phage Display Library Construction

Total RNA was extracted from the isolated lymphocytes using TRIZOL reagent (Thermo Fisher, Cata #.15596026) according to the manufacturer's protocol, which was used as starting material for RT-PCR to amplify sdAb encoding gene fragments. For gene amplification, the isolated RNA was reverse transcribed into cDNA with an oligo (dT)₂₀ primer using PRIMESCRIPT 1^{st} strand cDNA synthesis kit (Takara; catalog #: 6110A) according to the manufacturer's protocol. Six forward and two reverse specific degenerate primers were designed to amplify the V_{H}H fragments.

The variable regions of the V_{H}H fragments were amplified by two-step PCR methods. The DNA products of the first PCR were used as templates in the second PCR. The amplified second PCR products containing V_{H}H PCR fragments were gel purified and enzyme digested followed by insertion into phagemid plasmids. The recombinant plasmids were transferred into E. coli cells by electroporation, which generated the phage display library with size more than 1×10⁹. The library phage was prepared according to a standard protocol and stored after filter sterilization at −80° C. as stock.

### Phage Display Panning

The constructed phage library was either screened with CHO-K1 cells (ATCC; catalog #: CCL-61) overexpressing human CD16a cells (constructed by Genscript) or first counter-screened with CHO-K1 cells overexpressing human CD16b cells (constructed by Genscript) followed by panning with CHO-K1 cells overexpressing human CD16a using a standard procedure developed by GenScript. At least two rounds of selections were performed and each selection output was analyzed for enrichment factor (number of phage present in elution relative to control), diversity and percentage of CD16a positive CD16b negative or weak clones.

Based on these parameters the best selections were chosen for further screening, which was subcloned as a pool into a soluble expression vector for high-throughput screening. In frame with the sdAb coding sequence, the vector coded for a C-terminal His-Tag. Colonies were picked and grown in 96 deep well plates (1 ml volume) and induced by adding IPTG and 0.1% Triton for sdAb expression in the supernatant. The supernatants were screened through enzyme-linked immunosorbent assay (ELISA). Briefly, 96-well plate was coated with 0.5 ug/ml CD16a & CD16b protein respectively and was incubated at 4° C. for 16 hours, followed by being blocked with 3% MPBS. Primary antibody incubation was performed by adding 50 ul supernatant to each well and the plate was incubated at 37° C. for 1 hour. After that, 100 ul anti-V_{H}H secondary antibody (Genscript; catalog #: A01861) were pipetted into each well. Then the plate was incubated at room temperature (RT) for 60 min. After washing using 0.05% PBST, 100 ul TMB were added to each well. The plate was then incubated at RT for 15 min., and 50 ul 1M HCL were added to stop the reaction. The plate was read at 450 nm.

Production of sdAb-Fc Fusion Proteins

The anti-CD16a sdAb-Fc fusion protein constructs were generated by fusion of anti-CD16a sdAbs with human IgG4 (or mouse IgG1) Fc region. The maxiprep of the constructs were prepared for CHO-3E7 cell transient expression. The expressed anti-CD16a sdAb-Fc fusion proteins were purified by chromatography through a column containing Protein A agarose resin. 22 molecules were yielded.

### Example 2: Characterization of Anti-CD16a sdAbs In Vitro

The Specific Affinity of Anti-CD16a sdAbs to CD16a

As discussed above, Humans CD16 can be expressed in two isoforms, CD16a and CD16b, which are highly homologous, sharing 96% sequence identity in their extracellular immunoglobulin-binding regions. Moreover, CD16b can exist as a soluble receptor in serum and, upon binding to antibodies, may trigger side-effects via the formation of immune complexes in vivo. An antibody specific for CD16a would be particularly valuable in constructing bispecific or multispecific antibodies that are directed against disease-associated cells. In this case, the antibodies would mainly recruit NK cells and would not be bound by circulating soluble CD16b or diverted from NK cells binding by binding to neutrophils or activated eosinophils.

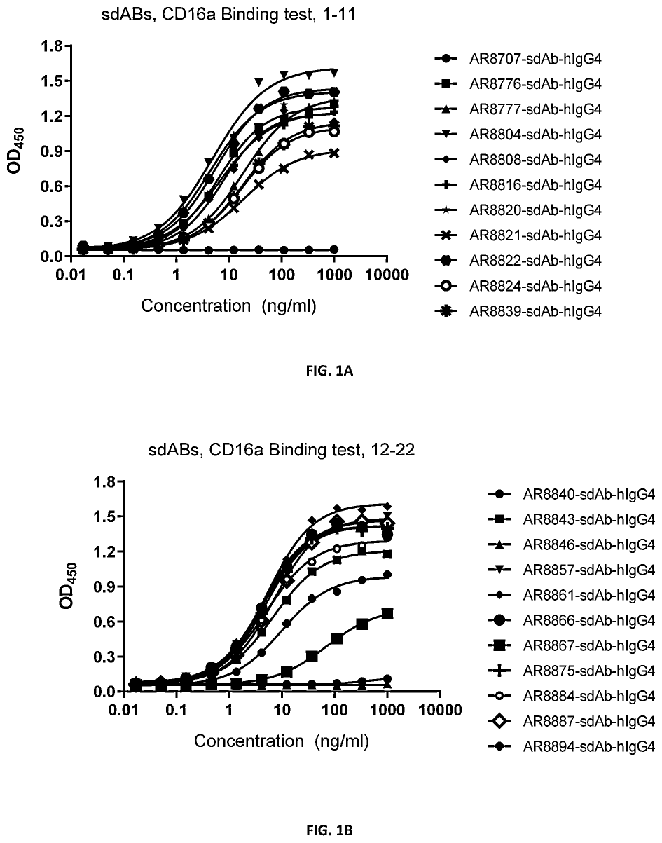

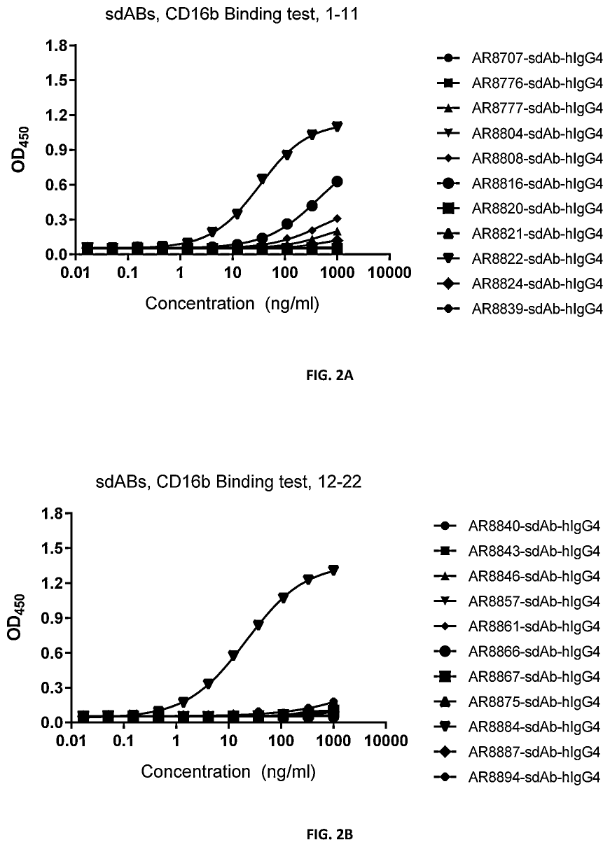

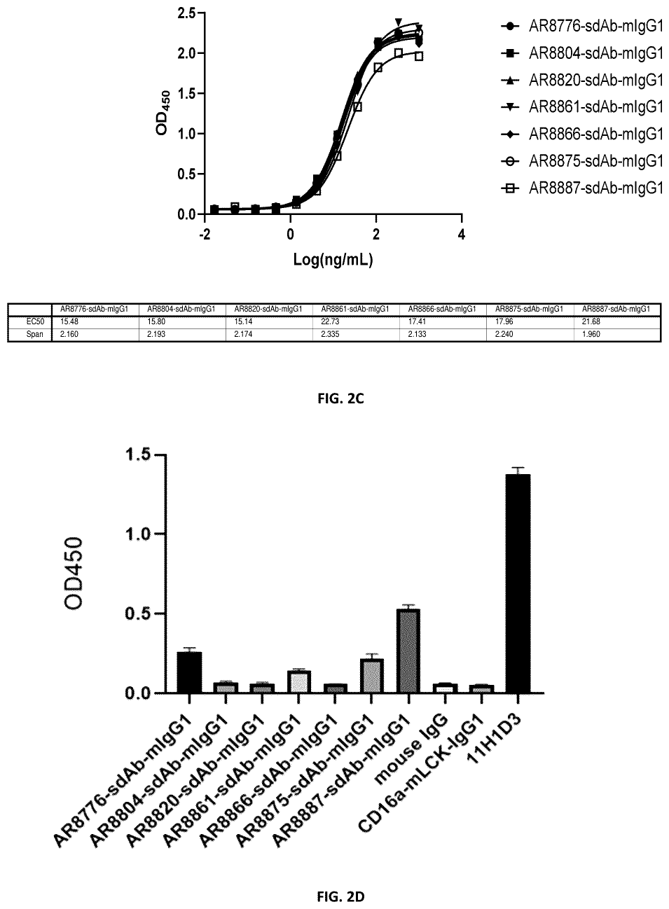

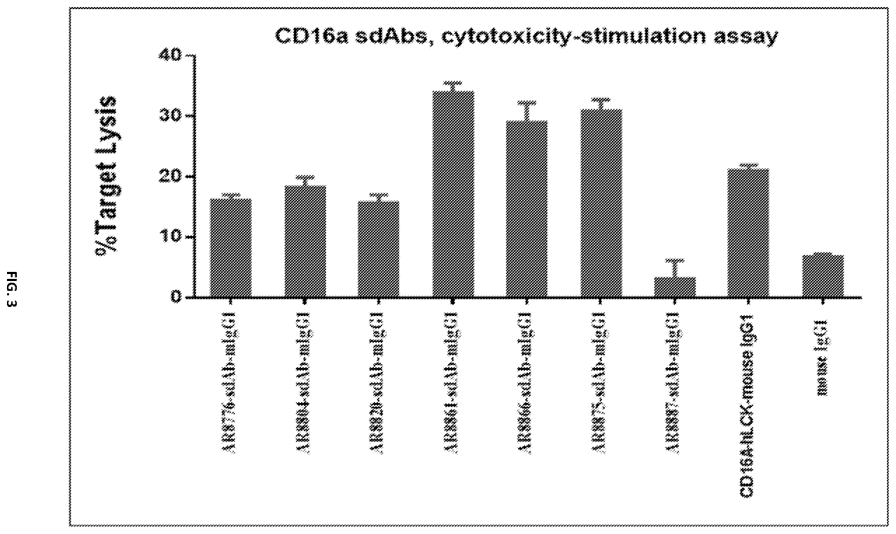

To determine the binding specificity of the 22 sdAbs to human CD16a but not to CD16b, human CD16a (Acro Biosystems; catalog #: CDA-H52S1) or CD16b (Acro Biosystems; catalog #: CDB-H5227) was pre-coated onto ELISA plates (0.5 μg/ml, 100 μl) overnight at 4° C. On the next day, wells were incubated with 3× serial diluted sdAbs fusion proteins (with hIgG4 Fc region, SEQ ID NO:73), with initial concentration of 1.0 μg/ml, following with HRP-conjugated goat anti human IgG (H+L) (Rockland) for substrate TMB chromogenic reaction. The bindings were quantified with optical densities readings at 450 nm. The sdAbs that showed specific affinity to human CD16a, but no or negligibly low affinity to CD16b were chosen for further screenings. According to the results (FIG. 1A, FIG. 1B, FIG. 2A, FIG. 2B), 7 sdAb molecules in total, namely AR8804, AR8820, AR8776, AR8875, AR8866, AR8887, AR8861 (SEQ ID NO: 1, 5, 9, 13, 17, 21, 25), were chosen for further verification. These selected leads were then fused to mouse IgG1 Fc for further study.

### Example 3: The sdAbs' NK Cytotoxicity-Triggering Potency

In this part, AR8776, AR8804, AR8820, AR8861, AR8866, AR8875 and AR8887 sdAbs were relinked to mouse IgG1 Fc format (SEQ ID NO:74), and their affinity specificity for CD16a were verified through ELISA. Briefly, for CD16a binding assay, human CD16a (Acro Biosystems; catalog #: CDA-H52S1) was pre-coated onto ELISA plates (1 μg/ml, 100 μl) overnight at 4° C. On the next day, wells were incubated with 3× serial diluted sdAbs fusion proteins (with mIgG1 Fc), with initial concentration of 1.0 μg/ml, followed by incubation with HRP-conjugated goat anti-mouse IgG (H+L) (Rockland; catalog #: 610-103-121) for substrate TMB chromogenic reaction. For CD16b binding assay, CD16b (Acro Biosystems; catalog #: CDB-H5227) was pre-coated onto ELISA plates (1 μg/ml, 100 μl) overnight at 4° C. On the next day, wells were incubated with sdAbs fusion proteins (with mIgG1 Fc) at single dose of 1.0 μg/ml, followed by incubation with HRP-conjugated goat anti-mouse IgG (H+L) (Rockland; catalog #: 610-103-121) for substrate TMB chromogenic reaction. The relinked molecules retained the affinity specificity to CD16a (FIG. 2C, FIG. 2D). AFM-CD16a-mIgG1 (also written as CD16a-mLCK-IgG1 or AFM13-CD16a-mIgG1) were used as reference control sample, while mouse IgG as isotype control. 11H1D3, an in-house mAb-mouse IgG1 antibody against human CD16a that proved to have high affinity with human CD16b, was exploited as positive sample in this experiment. The ELISA experiment was followed by reverse ADCC assay to gauge the strength of these molecules to trigger NK cells' cytotoxicity. Briefly, 10,000 human NK-92 158V/V cells were cocultured with mouse P815 target cells (SailyBio, Shanghai) pre-opsonized with 5 μg/ml sdAbs at E/T ratio of 5:1 for 4 H. Afterwards, the percentage of specific lysis was calculated with LDH cytotoxicity kit (Roche). AFM-CD16a-mIgG1 (also written as CD16a-mLCK-IgG1) were used as reference control sample, while mouse IgG as isotype control. The results indicated that all the sdAbs except AR8887 showed significant cytotoxicity-triggering activity, while, among them, molecules AR8861, AR8866, and AR8875 manifested superior cytotoxicity-triggering activity to the reference antibody (FIG. 3). So, molecules of AR8804, AR8820, AR8861, AR8866 and AR8875 were selected for humanization.

### Example 4. Humanization Antibody Production and Analysis

### Humanization Design for the Candidate Antibodies

Based on antibodies' potency in function assays and their variable domain sequences, the CDRs, HV loops and FRs were analyzed and homology modeling was performed to obtain the modeled structure of the sdAbs of AR8804, AR8820, AR8861, AR8866 and AR8875. The solvent accessible surface area of framework residues was calculated. Based on the result, framework residues that are buried (i.e. with solvent accessible surface area of <15%) were identified. several human acceptors for VH that share the top sequences identical to the llama counterparts were selected. The CDRs of the llama antibody were directly grafted to the human acceptor frameworks to obtain the sequence of the grafted antibody. Post translational modifications and chemical degradation in grafted sequence including deamidation, isomerization oxidation and glycosylation, etc., were analyzed through developability assessment. PTM hotspots like N-glycosylation sites, unusual proline residues, deamidation site, isomerization site, oxidation site and unpaired cysteine residues etc. that may affect the binding activity and manufacturability of the grafted antibody, were identified. The DNA sequences encoding the humanized antibodies were synthesized. The antibody characteristics were compared to select the best candidate. These humanized molecules were successfully produced.

### Humanized Antibody Production

For humanized antibody production, we fused the humanized anti-CD16a sdAbs to the N-terminus of human IgG4 (or mouse IgG1) Fc region to generate anti-CD16a sdAb-Fc fusion proteins. The DNA sequence expressing each anti-CD16a sdAb-Fc fusion protein was inserted into pTT5 vector between EcoRI and HindIII restriction sites. CHO-3E7 cells transfected with these expression plasmids were cultured at 37° C. and 100 rpm for 6 days. The supernatant fraction was collected by centrifugation and the proteins were purified through Protein A column.

### Humanized Antibody Specific Binding Affinity Analysis ELISA

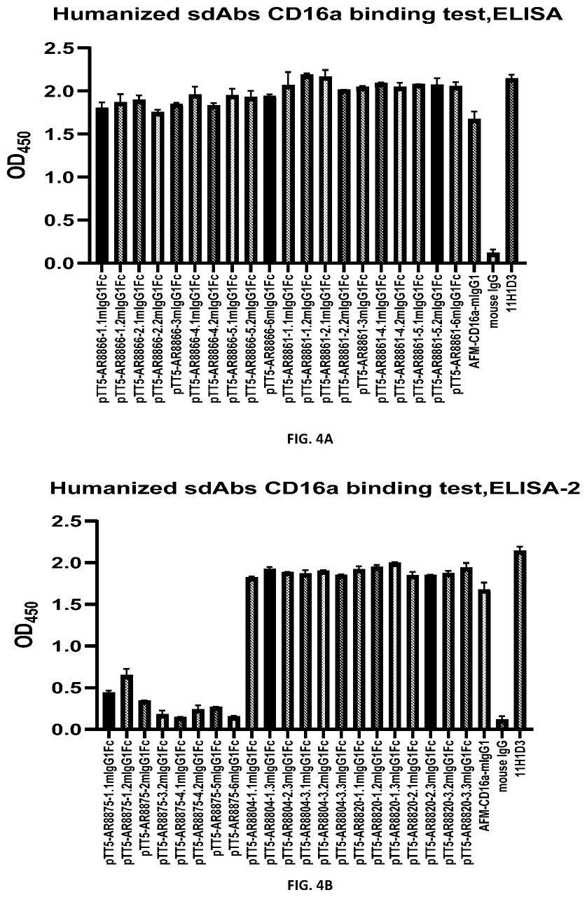

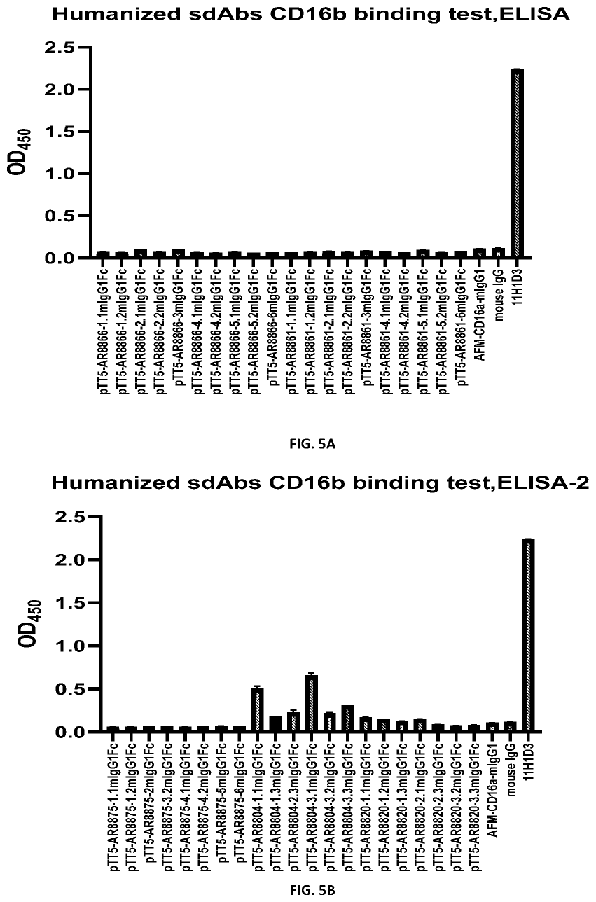

As discussed above, an antibody specific for CD16a would be particularly valuable in constructing bispecific or multispecific antibodies that are directed against disease-associated cells. To determine protein binding specificity of humanized sdAbs by ELISA, 41 purified humanized sdAbs-mIgG1 Fc fusion proteins derived were pre-coated onto ELISA plates (0.5 μg/ml, 100 μl) overnight at 4° C. On the next day, wells were incubated with 1.0 μg/ml primary antibodies, following with HRP-conjugated goat anti-mouse IgG (H+L) (Rockland; catalog #: 610-103-121) for substrate TMB chromogenic reaction. Antibody-CD16a and CD16b bindings were quantified with optical densities readings at 450 nm (FIGS. 4A-4B, FIGS. 5A-5B), and candidates having specific affinity to CD16a, were selected for further screenings. AFM-CD16a-mIgG1 were used as reference control sample, while mouse IgG as isotype control.

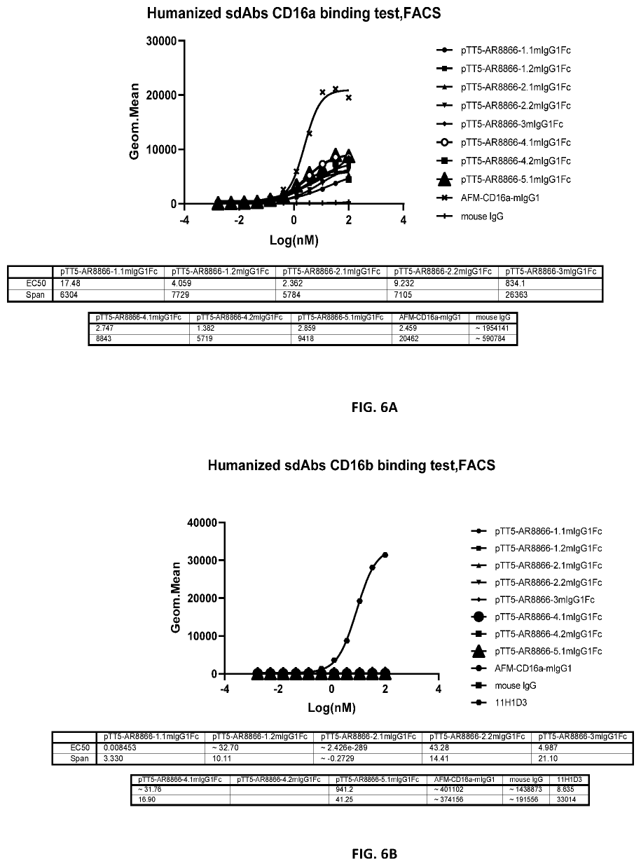

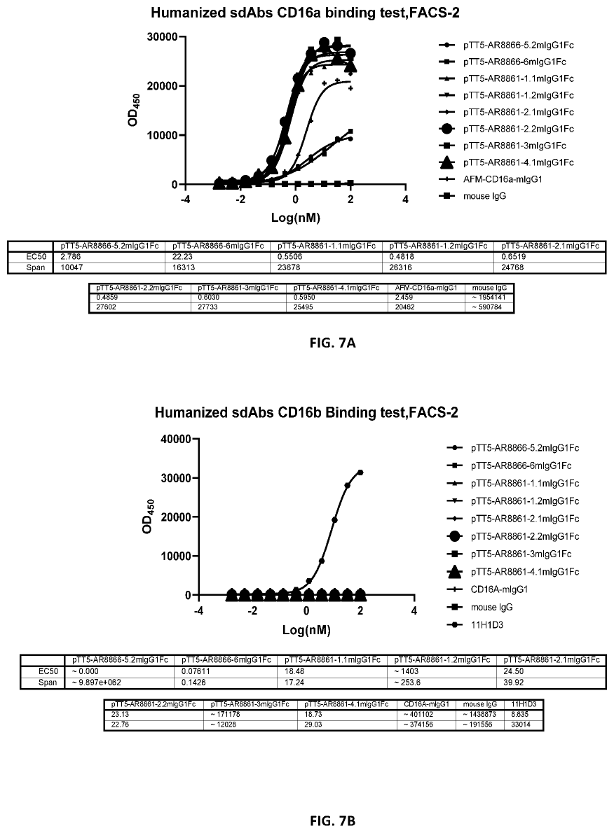

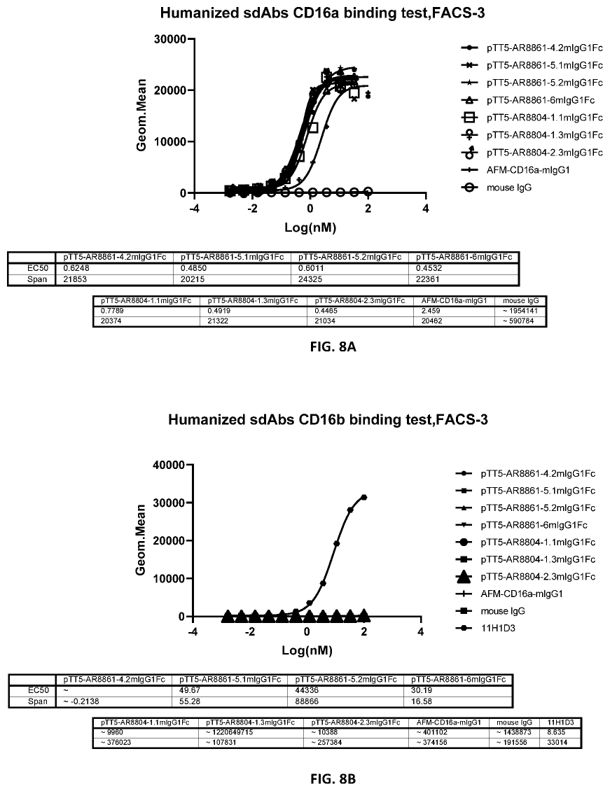

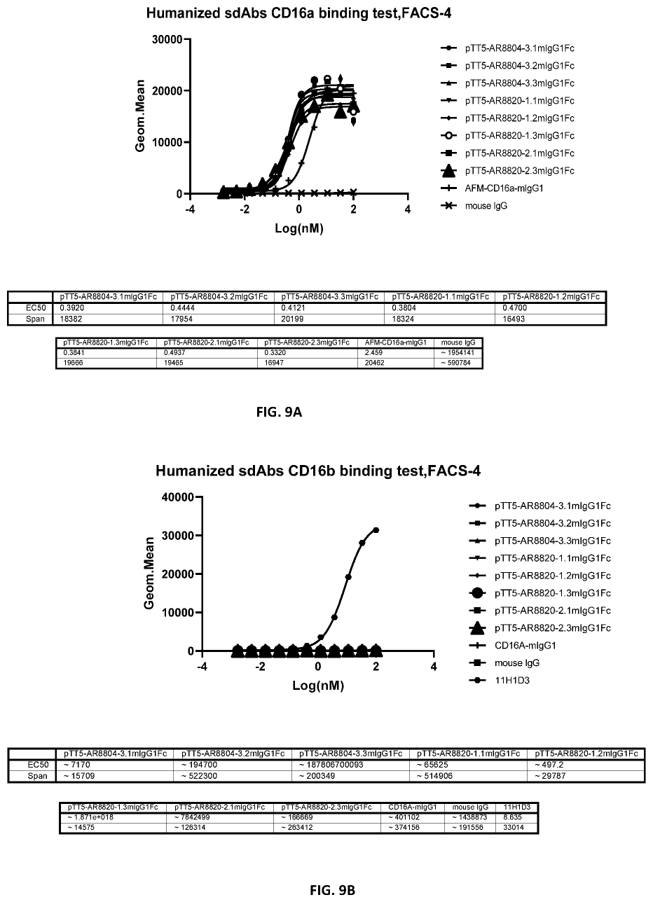

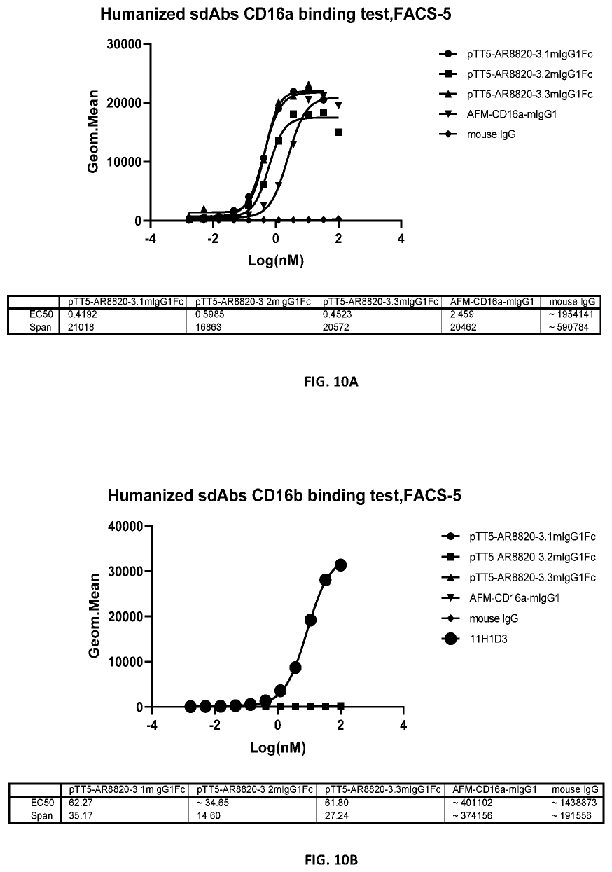

Humanized molecules of significant affinity to human CD16a but of no or negligible affinity to human CD16b, 34 in total, were chosen for further confirmation by FACS. Briefly, to determine cell surface CD16a binding EC₅₀ of the sdAb products, HEK293 cells expressing human CD16a (constructed by Genscript) were harvested and incubated with serially diluted anti-CD16a sdAbs (max. concentration: 100 nM, 3-fold dilution), followed by fluorophore (iFluor 647)-labeled goat anti-mouse IgG (H+L) secondary antibodies (Jackson immuno research; catalog #: 115-605-062). The samples were then analyzed with flow cytometry. Raw data was plotted with GraphPad Prism v6.02 software with four parameters, best-fit values program to analyze the EC₅₀. AFM-CD16a-mIgG1 (CD16a-mIgG1) was used as reference control sample, while mouse IgG as isotype control. The results (FIGS. 6A/6B/7A/7B/8A/8B/9A/9B/10A/10B) confirmed the affinity specificity of the chosen molecules. However, all derivatives from molecule AR8866, 10 in total, showed much weaker affinity than those from other parental molecules.

The Humanized sdAbs' NK Cytotoxicity-Triggering Potency

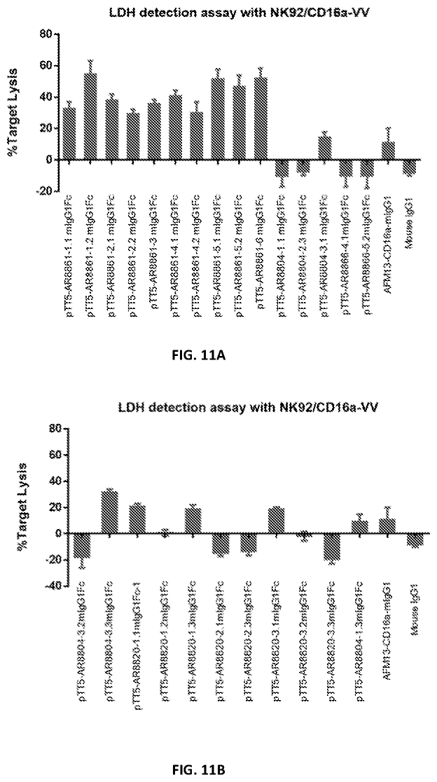

The strength of the humanized sdAbs to trigger NK cells' cytotoxicity were gauged by way of reverse ADCC assay as described above. Briefly, 10,000 human NK-92 158V/V cells were cocultured with mouse P815 target cells (SailyBio, Shanghai) pre-opsonized with 0.008 μg/ml sdAbs-mIgG1 Fc fusion protein (pTT5-humanized sdAb-mIgG1Fc) at E/T ratio of 5:1 for 4 hours. Afterwards, the percentage of specific lysis was calculated with LDH cytotoxicity kit (Roche). AFM13-CD16a-mIgG1 was used as reference control, while mouse IgG as isotype control. As shown in FIGS. 11A and 11B, the results indicated that sixteen molecules including pTT5-AR8861-1.1mIgG1Fc, pTT5-AR8861-1.2mIgG1Fc, pTT5-AR8861-2.1mIgG1Fc, pTT5-AR8861-2.2mIgG1Fc, pTT5-AR8861-1.3mIgG1Fc, pTT5-AR8861-4.1mIgG1Fc, pTT5-AR8861-4.2mIgG1Fc, pTT5-AR8861-5.1mIgG1Fc, pTT5-AR8861-5.2mIgG1Fc, pTT5-AR8861-6mIgG1Fc, pTT5-AR8804-3.1 mIgG1Fc, pTT5-AR8804-3.3mIgG1Fc, pTT5-AR8804-1.3 mIgG1Fc, pTT5-AR8820-1.1mIgG1Fc, pTT5-AR8820-1.3mIgG1Fc, and pTT5-AR8820-3.1 mIgG1Fc, exhibited significant potency in triggering NK cell cytotoxicity (FIGS. 11A/11B).

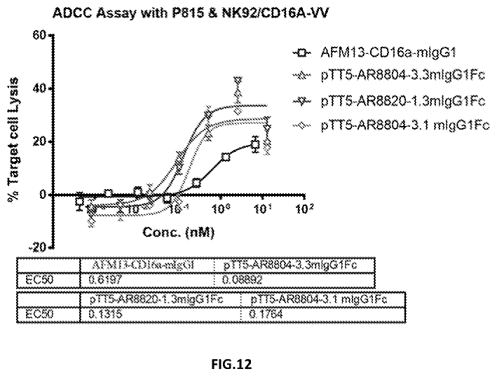

To further confirm the in vitro activity, some of these anti-human CD16a sdAb leads were selected for dose dependent response of reverse ADCC bioassay. Briefly, 10,000 human NK-92 158V/V cells were cocultured with mouse P815 target cells (SailyBio, Shanghai) pre-opsonized with 5× serial diluted samples (diluted from the initial concentration of 1.0 μg/ml) at E/T ratio of 5:1 for 4 H. Afterwards, the percentage of specific lysis was calculated with LDH cytotoxicity kit (Roche). As shown in FIG. 12, the selected leads of pTT5-AR8804-3.1 mIgG1Fc, pTT5-AR8804-3.3mIgG1Fc and pTT5-AR8820-1.3mIgG1Fc exhibited much higher activity than the reference antibody of AFM13-CD16a-mIgG1.

### The Cross-Species Reactions of Anti-CD16a Humanized Antibodies With Cynomolgus Monkey CD16a Protein

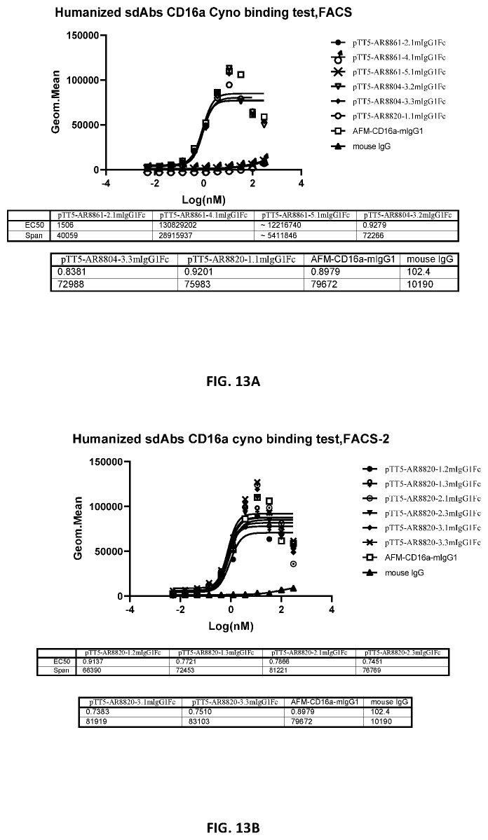

To determine the cross-reactions of antibody products, HEK293 cells expressing cyno monkey CD16a (constructed by Genscript) were harvested and incubated respectively with serially diluted anti-CD16a humanized sdAb-mIgG1Fcs (max. concentration: 100 nM, 3-fold dilution), followed by fluorophore (iFluor 647)-labeled goat anti mouse IgG (H+L) secondary antibodies (Jackson immuno research; catalog #: 115-605-062). The samples were then analyzed with flow cytometry. Raw data was plotted with GraphPad Prism v6.02 software with four parameters, best-fit values program to analyze the EC₅₀. The molecules of pTT5-AR8804-3.2 mIgG1Fc, pTT5-AR8804-3.3mIgG1Fc, pTT5-AR8820-1.1mIgG1Fc, pTT5-AR8820-1.2mIgG1Fc, pTT5-AR8820-1.3mIgG1Fc, pTT5-AR8820-2.1mIgG1Fc, pTT5-AR8820-2.3mIgG1Fc, pTT5-AR8820-3.1mIgG1Fc and pTT5-AR8820-3.3 mIgG1Fc manifested good cross-reaction with cynomolgus monkey CD16a protein (FIG. 13).

1. An isolated antibody, or an antigen-binding portion thereof, comprising:
a monomeric variable domain comprising a CDR1 region, a CDR2 region, and a CDR3 region comprising the amino acid sequences of:
SEQ ID NOs: 2, 3, and 4, respectively; or
SEQ ID NOs: 6, 7, and 8, respectively; or
SEQ ID NOs: 14, 15, and 16, respectively; or
SEQ ID NOs: 14, 15, and 64, respectively; or
SEQ ID NOs: 14, 15, and 66, respectively; or
SEQ ID NOs: 18, 19, and 20, respectively; or
SEQ ID NOs: 26, 27, and 28, respectively; or
a variant thereof comprising up to about 3 amino acid substitutions in any one or more of the CDR1 region, the CDR2 region, and the CDR3 region.

2. (canceled)

3. The antibody or antigen-binding portion thereof, of claim 1, which specifically binds to human CD16a.

4. The antibody or antigen-binding portion thereof, of claim 1, which is a single domain antibody (sdAb) or a V_{H}H domain.

5. The antibody or antigen-binding portion thereof, of claim 1, which is camelid, chimeric, human or humanized.

6. The antibody or antigen-binding portion thereof, of claim 1,wherein the monomeric variable domain comprises an amino acid sequence having at least 80%, 85%, 90%, 92%, 94%, 95%, 96%, 97%, 98%, or 99% identity to the amino acid sequence selected from the group consisting of SEQ ID NOs: 44, 47, 1, 5, 13, 17, 25, 29, 30, 31, 32, 33, 34, 35, 36, 37, 38, 39, 40, 41, 42, 43, 45, 46, 48, 49, 50, 51, 52, 53, 54, 55, 56, 57, 58, 59, 60, 61, and 62.

7. The antibody or antigen-binding portion thereof, of claim 6, wherein the monomeric variable domain comprises an amino acid sequence selected from the group consisting of SEQ ID NOs: 44, 47, 1, 5, 13, 17, 25, 29, 30, 31, 32, 33, 34, 35, 36, 37, 38, 39, 40, 41, 42, 43, 45, 46, 48, 49, 50, 51, 52, 53, 54, 55, 56, 57, 58, 59, 60, 61, and 62.

8.-9. (canceled)

10. The antibody or antigen-binding portion thereof, of claim 1, wherein the monomeric variable domain comprises an amino acid sequence having at least 80%, 85%, 90%, 92%, 94%, 95%, 96%, 97%, 98%, or 99% identity to the amino acid sequence selected from the group consisting of SEQ ID NOs: 44, 47, 42, 45, and 50.

11. The antibody or antigen-binding portion thereof, of claim 10, wherein the monomeric variable domain comprises an amino acid sequence selected from the group consisting of SEQ ID NOs: 44, 47, 42, 45, and 50.

12. An antibody or an antigen-binding portion thereof, comprising a monomeric variable domain comprising an amino acid sequence selected from the group consisting of SEQ ID NOs: 44, 47, 1, 5, 13, 17, 25, 29, 30, 31, 32, 33, 34, 35, 36, 37, 38, 39, 40, 41, 42, 43, 45, 46, 48, 49, 50, 51, 5253, 54, 55, 56, 57, 58, 59, 60, 61, and 62.

13. The antibody or antigen-binding portion thereof, of claim 12, wherein the antibody is a humanized antibody, and the monomeric variable domain comprises the amino acid sequence selected from the group consisting of SEQ ID NOs: 44, 47, 42, 45, and 50.

14. The antibody or antigen-binding portion thereof, of claim 1, further comprising an IgG Fc region fused with the monomeric variable domain.

15. The antibody or antigen-binding portion thereof, of claim 14, wherein the IgG Fc region is a human IgG4 Fc region or a mouse IgG1 Fc region.

16. A bispecific molecule, an immunoconjugate, or a chimeric antigen receptor, comprising the antibody or antigen-binding portion thereof of claim 1.

17. A nucleic acid molecule encoding the antibody or antigen-binding portion thereof of claim 1.

18. An expression vector containing the nucleic acid molecule of claim 17.

19. A host cell containing the expression vector of claim 18.

20. A pharmaceutical composition comprising the antibody or antigen-binding portion thereof, of claim 1 and a pharmaceutically acceptable carrier.

21. (canceled)

22. A method for treating a cancer disease in a subject, comprising administering to the subject a therapeutically effective amount of the pharmaceutical composition of claim 20.

23. The method of claim 22, wherein the cancer disease is selected from the group consisting of adrenal cancer, bladder cancer, breast cancer, colorectal cancer, gastric cancer, glioblastoma, kidney cancer, non-small-cell lung cancer, acute lymphocytic leukemia, acute myeloid leukemia, chronic lymphocytic leukemia, chronic myeloid leukemia, hairy cell leukemia, Burkett's lymphoma, diffuse large B cell lymphoma, follicular lymphoma, mantle cell lymphoma, marginal zone lymphoma, non-Hodgkin's lymphoma, small lymphocytic lymphoma, multiple myeloma, melanoma, ovarian cancer, pancreatic cancer, prostate cancer, skin cancer, renal cell carcinoma, testicular cancer, and uterine cancer.

24. A method of promoting NK cell proliferation in a subject, comprising administering to the subject a therapeutically effective amount of the pharmaceutical composition of claim 20.
